(19) United States
(12) Patent Application Publication
(10) Pub. No.: US 2022/0330899 A1
(43) Pub. Date: Oct. 20, 2022

(54) DETECTION AND PREDICTION OF HYPERTENSION INDUCED ORGAN DAMAGE USING ECG AND BLOOD PRESSURE DATA

(71) Applicant: AliveCor, Inc., Mountain View, CA (US)

(72) Inventor: Archana Dubey, Palo Alto, CA (US)

(21) Appl. No.: 17/231,909

(22) Filed: Apr. 15, 2021

(51) Int. Cl. A61B 5/00; A61B 5/346; A61B 5/021

(52) U.S. Cl. CPC A61B 5/7275; A61B 5/346; A61B 5/021; A61B 5/7264; A61B 5/4836; A61B 5/4848

(57) ABSTRACT

Embodiments of the present disclosure provide systems and methods for diagnosing LVH based on a user's ECG data as well as blood pressure data. The user may record their ECG and blood pressure data using any appropriate ECG and blood pressure monitors, and may augment the ECG and blood pressure data with user characteristics such as user age, sex, diet, and previous medical history before transmitting the ECG and blood pressure data to a cloud storage system. A machine learning (ML) model implemented in the cloud storage system may analyze ECG data of the user using LVH diagnosis criteria, and augment the results of the ECG data analysis with the blood pressure data of the user to form a diagnosis. The diagnosis may indicate whether the user is suffering from LVH, as well as a severity of the LVH.

### TECHNICAL FIELD

Aspects of the present disclosure relate to detection of left ventricular hypertrophy (LVH), and in particular to detecting LVH in a user based on the user's electrocardiogram (ECG) data and blood pressure data.

### BACKGROUND

Cardiovascular diseases are the leading cause of death in the world. For example, in 2008 approximately 30% of all deaths globally may have been attributed to cardiovascular diseases. It is also estimated that by 2030, there will be over 23 million cardiovascular disease related deaths annually. Cardiovascular diseases are prevalent in various demographics. In the general population, the left ventricle (LV) of the heart may remodel over the course of a person's life as an adaptive response to a variety of non-modifiable and modifiable cardiovascular risk factors such as age, sex, ethnicity, hypertension, and underlying medical conditions, among others.

Systemic hypertension (also referred to as blood pressure) is one of the most important risk factors involved in the transition process from a normal structure/geometry of the LV to a remodeled structure/geometry of the LV due to myocyte hypertrophy and interstitial fibrosis resulting in alterations of both LV contractility and relaxation. These morpho-functional alterations are the substrate of the so-called hypertensive heart disease. In particular, LV hypertrophy (LVH), routinely assessed by electrocardiography or, more accurately, by echocardiography, is the pivotal biomarker of subclinical cardiac damage. LVH represents an intermediate stage in the continuum of cardiovascular disease linking risk factors and cardiovascular fatal and nonfatal events. A solid body of evidence supports the view that LVH is a powerful predictor of cardiovascular disease over and beyond traditional risk factors and that its regression is associated with an improvement in cardiovascular prognosis.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a block diagram that illustrates an example system, in accordance with some embodiments of the present disclosure.

FIG. 2 is a diagram illustrating various leads that an ECG can be comprised of, in accordance with some embodiments of the present disclosure.

FIG. 3 is a block diagram that illustrates a cloud storage system that may store ECG and blood pressure data of various users, in accordance with some embodiments of the present disclosure.

FIG. 4A is a diagram that illustrates example measurements of the various leads illustrated in FIG. 2, in accordance with some embodiments of the present disclosure.

FIG. 4B is a block diagram that illustrates an example convolutional neural network (CNN), in accordance with some embodiments of the present disclosure.

FIG. 4C is a block diagram that illustrates an example training process for a CNN, in accordance with some embodiments of the present disclosure.

FIG. 5 is a flow diagram of a method for detecting LVH, in accordance with some embodiments of the present disclosure.

FIG. 6 is a flow diagram of a method of identifying optimal courses of treatment for reducing LVH, in accordance with some embodiments of the present disclosure.

FIG. 7 is a block diagram of an example computing device that may perform one or more of the operations described herein, in accordance with some embodiments of the present disclosure.

### DETAILED DESCRIPTION

The risk of developing LVH (among other cardiological conditions) has been shown to depend on the severity of hypertension, that is, to progressively increase as blood pressure increases from the upper normal limit of 140/90 mm Hg. Evidence is also available that the relationship between blood pressure and certain cardiovascular conditions persists at blood pressure values less than 140/90 mm Hg. Individuals with a blood pressure in the so-called prehypertension range (e.g., 120-139/80-89 mm Hg) show a distinctly greater cardiovascular risk than those with lower blood pressure values, that is, less than 120/80 mm Hg. Various cross-sectional studies have dealt with the association between prehypertension and subclinical cardiac damage, including the development of LVH over time when prehypertension is present.

Because hypertension is asymptomatic, diagnosis of this condition is often delayed, as it depends on effective user (e.g., patient) and provider engagement. This may lead to organ damage from complications of hypertension, such as LVH (which may be found in 23-48% of users with hypertension). LVH in turn may lead to an increased risk of coronary artery disease (CAD), atrial fibrillation (Afib) and other arrhythmias, and congestive heart failure (CHF). In many instances, LVH is left undiagnosed due to dependency on the provider to do an ECG or echocardiogram (referred to herein as echo) on the user. LVH is largely diagnosed via Echo, for example as a work up for EF, palpitation for arrhythmia or stress echo for CAD, or when an ECG is done in the physician's office as a work up for other cardiac symptoms, as an incidental finding. As a result, even when ECG has been used to diagnose LVH in users, such diagnosis is performed independently of and does not account for blood pressure data, which has been shown to be an important indicator of the presence of LVH (as well as other cardiac conditions).

The present disclosure addresses the above-noted and other deficiencies by providing systems and methods for diagnosing LVH based on a user's ECG data as well as blood pressure data. The user may record their ECG and blood pressure data using any appropriate ECG and blood pressure monitors, and may augment the ECG and blood pressure data with user characteristics such as user age, sex, diet, and previous medical history before transmitting the ECG and blood pressure data to a cloud storage system. A machine learning (ML) model implemented in the cloud storage system may be used to analyze ECG data of the user using LVH diagnosis criteria (also referred to herein as ECG criteria), and to augment the results of the ECG data analysis with the blood pressure data of the user to form a diagnosis. In some embodiments, the ML model may further augment the diagnosis based on user characteristics such as user age, sex, diet, and previous medical history. The diagnosis may indicate whether the user is suffering from LVH, as well as a severity of the LVH.

Upon generating the diagnosis, a physician or computer system may prescribe a course of treatment for the user, who may continue to record their ECG and blood pressure data during the course of treatment. The ML model may monitor the ECG and blood pressure data recorded during this time to determine if an improvement or worsening of their LVH has occurred. Simultaneously, the ML model may monitor the ECG and blood pressure data of a variety of other users undergoing any of a plurality of courses of treatment for LVH. Over time, the ML model may learn what courses of treatment work for different users based on user characteristics such as user age, sex, diet, and previous medical history. Based on this, the ML model may begin prescribing courses of treatment for users who are diagnosed with LVH by matching them with courses of treatment that have worked for users with similar user characteristics. Notably, although the terms “user,” “physician,” and “provider” are used throughout for convenience, the embodiments described herein are equally suitable in the context of a single user, without input from a physician or other third party.

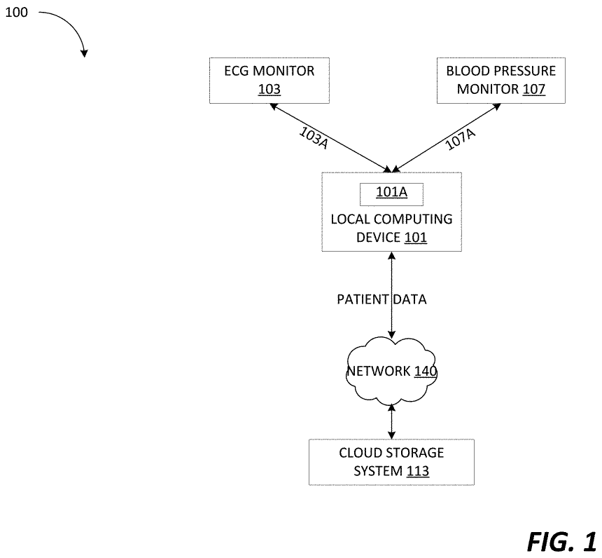

FIG. 1 shows a system 100 for cardiac disease management. The system 100 may be prescribed for use by a first user e.g., by the first user's physician. Alternatively, system 100 may be used without input from a physician or other third party. The system 100 may comprise a local computing device 101 of the first user. The local computing device 101 may be loaded with a user interface, dashboard, or other sub-system of the cardiac disease management system 100. For example, the local computing device 101 may be loaded with a mobile software application (“mobile app”) 101A for interfacing with the system 100. The mobile app 101A may be configured to interface with one or more biometric sensors (e.g., ECG monitor 103) and may comprise software and a user interface for managing biometric data collected by the local computing device 101 from one or more biometric sensors. The local computing device 101 may comprise any appropriate computing device, such as a tablet computer, a smartphone, a server computer, a desktop computer, a laptop computer, or a body-worn computing device (e.g., a smart watch or other wearable), for example. In some embodiments, the local computing device 101 may comprise a single computing device or may include multiple interconnected computing devices (e.g., multiple servers configured in a cluster).

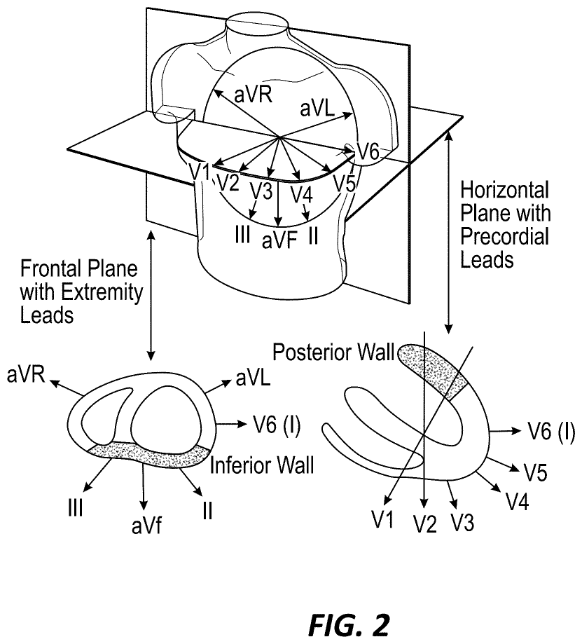

The local computing device 101 may be coupled to one or more biometric sensors. For example, the local computing device 101 may be coupled to an ECG monitor 103 which may comprise a set of electrodes for recording ECG (electrocardiogram) data (also referred to herein as “taking an ECG”) of the first user's heart. The ECG data can be recorded or taken using the set of electrodes which are placed on the skin of the first user in multiple locations. The electrical signals recorded between electrode pairs may be referred to as leads and FIG. 2 illustrates a 12 lead set comprising the I, II, III, aVR, aVL, aVF, V1, V2, V3, V4, V5, and V6 leads, all represented on a hexaxial system. Varying numbers of leads can be used to record the ECG data, and different numbers and combinations of electrodes can be used to form the various leads. Example numbers of leads used for taking ECGs are 1, 2, 6, and 12 leads. For example, the ECG monitor 103 may be a device comprising 10 electrodes (with six on the user's chest and one on each of the user's arms and legs) which may provide a 12-lead ECG. The electrode placed on the right arm may be referred to as RA. The electrode placed on the left arm may be referred to as LA. The RA and LA electrodes may be placed at the same location on the left and right arms, e.g., near the wrist. The leg electrodes may be referred to as RL for the right leg and LL for the left leg. The RL and LL electrodes may be placed on the same location for the left and right legs, e.g., near the ankle.

In some embodiments, the ECG monitor 103 may comprise a handheld ECG monitor (such as the KardiaMobile® or KardiaMobile® 6L device from AliveCor® Inc., for example) comprising a smaller number of electrodes (e.g., 2 or 3 electrodes). In these embodiments, the electrodes can be used to measure a subset of the leads illustrated in FIG. 2, such as lead I (e.g., the voltage between the left arm and right arm) contemporaneously with lead II (e.g., the voltage between the left leg and right arm), and lead I contemporaneously with lead V2 or another one of the chest leads such as V5. It should be noted that any other combination of leads is possible. If desired, additional leads can then be algorithmically derived (e.g., by the ECG monitor 103 itself or the local computing device 101) from the determined subset of leads. For example, augmented limb leads can also be determined from the values measured by the LA, RA, LL, and RL electrodes. The augmented vector right (aVR) may be equal to RA−(LA+LL)/2 or −(I+II)/2. The augmented vector left (aVL) may be equal to LA−(RA+LL)/2 or I−II/2. The augmented vector foot (aVF) may be equal to LL−(RA+LA)/2 or II−I/2. In some embodiments, the ECG monitor 103 itself or the local computing device 101 may utilize a machine learning (ML) model to derive the full 12 lead set from a measured subset of leads. In some embodiments, the ECG monitor 103 may be in the form of a smartphone, or a wearable device such as a smart watch. In some embodiments, the ECG monitor 103 may be a handheld sensor coupled to the local computing device 101 with an intermediate protective case/adapter.

The ECG data recorded by the ECG monitor 103 may comprise the electrical activity of the first user's heart, for example. A typical heartbeat may include several variations of electrical potential, which may be classified into waves and complexes, including a P wave, a QRS complex, a T wave, and sometimes U wave as known in the art. The shape and duration of the P wave can be related to the size of the user's atrium (e.g., indicating atrial enlargement) and can be a first source of heartbeat characteristics unique to a user.

The QRS complex can correspond to the depolarization of the heart ventricles, and can be separated into three distinct waves—a Q wave, a R wave and a S wave. Because the ventricles contain more muscle mass than the atria, the QRS complex is larger than the P wave. Also, the His/Purkinje system of the heart, which can increase the conduction velocity to coordinate the depolarization of the ventricles, can cause the QRS complex to look “spiked” rather than rounded. The duration of the QRS complex of a healthy heart can be in the range of 60 to 100 milliseconds (ms), but can vary due to abnormalities of conduction. The duration of the QRS complex can serve as another source of heartbeat characteristics unique to a user.

The duration, amplitude, and morphology of each of the Q, R and S waves can vary in different individuals, and in particular can vary significantly for users having cardiac diseases or cardiac irregularities. For example, a Q wave that is greater than ⅓ of the height of the R wave, or greater than 40 ms in duration can be indicative of a myocardial infarction and provide a unique characteristic of the user's heart. Similarly, other healthy ratios of Q and R waves can be used to distinguish different users' heartbeats.

The electrical activity of the user US's heart can also include one or more characteristic durations or intervals that can be used to distinguish different users. For example, the electrical activity of the heart may include PR intervals and ST segments as known in the art. A PR interval can be measured from the beginning of P wave to the beginning of a QRS complex. A PR interval can typically last 120 to 200 ms. A PR interval having a different duration can indicate one or more defects in the heart, such as a first degree heart block (e.g., a PR interval lasting more than 200 ms), a pre-excitation syndrome via an accessory pathway that leads to early activation of the ventricles (e.g., a PR interval lasting less than 120 ms), or another type of heart block (e.g., a PR interval that is variable). An ST segment can be measured from a QRS complex to a T wave, for example starting at the junction between the QRS complex and the ST segment and ending at the beginning of the T wave. An ST segment can typically last from 80 to 120 ms, and normally has a slight upward concavity. The combination of the length of ST segment, and the concavity or elevation of ST segment can also be used to generate characteristic information unique to each user's heartbeat.

The ECG monitor 103 may be used by the first user to measure their ECG data and transmit the measured ECG data to the local computing device 101 by connection 103A as described in further detail herein. The connection 103A may comprise a wired or wireless connection (e.g., a Wi-Fi connection, a Bluetooth® connection, a near-field communication (NFC) connection, an ultrasound signal transmission connection, etc.).

The local computing device 101 may be coupled to other biometric devices as well such as a personal scale or a blood pressure monitor 107. The blood pressure monitor 107 may communicate with the local device 101 through a wired or wireless connection 107A (e.g., a Wi-Fi connection, a Bluetooth connection, an NFC connection, an ultrasound signal transmission connection, etc.). Although illustrated as separate devices, in some embodiments the ECG monitor 103 and the blood pressure sensor 107 may be implemented as a single device.

In some embodiments, the blood pressure monitor 107 may comprise a processor, an optical sensor, and a display (not shown in the FIGS.). In some embodiments, the optical sensor is a camera that operates at a minimum of 30 frames per second or at a minimum of 60 frames per second. The blood pressure monitor 107 may also comprise a non-transitory computer readable storage medium having a computer program including instructions executable by the processor to perform operations for measuring the blood pressure of first user. For example, the processor may receive a first ECG reading from a set of electrodes (e.g., the electrodes of the ECG monitor 103, or a separate dedicated set of electrodes) and simultaneously receive a first photoplethysmogram from the optical sensor. The processor may then receive a second ECG reading from the set of electrodes and simultaneously receive a second photoplethysmogram from the optical sensor and generate an average ECG reading from the first and second ECG readings. The processor may determine a differential pulse arrival time based on the average ECG reading and the first and second photoplethysmograms and determine said blood pressure of the user based on the differential pulse arrival time. The blood pressure data recorded by the blood pressure monitor 107 may comprise the systolic and diastolic blood pressure of the first user, for example.

In some embodiments, the blood pressure monitor 107 may be implemented on a smartphone, a tablet computer, laptop computer, a wearable device such as a smart watch, or any other appropriate computing device. In some embodiments where the blood pressure monitor 107 uses a dedicated set of electrodes, the dedicated set of electrodes may be removable from the blood pressure monitor 107. Although described as above, the blood pressure monitor 107 may comprise any appropriate hardware and may utilize any appropriate techniques for measuring blood pressure.

The ECG and blood pressure data may be continually recorded by the user at regular intervals. For example, the interval may be once a day, once a week, once a month, or some other predetermined interval. The ECG and blood pressure data may be recorded at the same or different times of days, under similar or different circumstances, as described herein. The ECG data and the blood pressure data may also be recorded at the same or different times of the interval (e.g., the ECG and blood pressure data may be captured asynchronously). Alternatively, or additionally, the ECG and blood pressure data can be recorded on demand by the user at various discrete times, such as when the user feels chest pains or experiences other unusual or abnormal feelings, or in response to an instruction to do so from e.g., the user's physician. In another embodiment, ECG or blood pressure data may be continuously recorded over a period of time (e.g., by a Holter monitor or by some other wearable device).

Each ECG and blood pressure data recording may be time stamped and may be annotated with additional data by the user or health care provider to describe user characteristics. For example, the local computing device 101 (e.g., the mobile app 101A thereof) may include a user interface for data entry that allows the user to enter their user characteristics. Examples of user characteristics may include age, sex, race, ethnicity, relevant medical history, location, diet (e.g., food/drink habits), medication/drug consumption, exercise patterns, sleep/rest patterns, feelings of stress, anxiety, pain or other unusual or abnormal feelings, activities performed before, during, or after the data recording, or any other user specific circumstance or factor that may affect the user's ECG and blood pressure data. The local computing device 101 may append the user characteristics to the ECG and blood pressure data and transmit the ECG data, blood pressure data, and user characteristics (collectively referred to as user data) to the cloud storage system 113. Because the user data is time stamped or tagged, the ECG and blood pressure data can be matched or correlated with an activity or circumstance of interest. As described in further detail herein, this also allows for comparison of the ECG and blood pressure data before, after and during the activity or circumstance of interest so that the effect on the ECG and blood pressure data can be determined and accounted for during further analysis.

The user data can be transmitted by the local computing device 101 to the cloud storage system 113 for storage and analysis. The transmission can be real-time, at regular intervals such as hourly, daily, weekly and/or any interval in between, or can be on demand. The local computing device 101 and the cloud storage system 113 may be coupled to each other (e.g., may be operatively coupled, communicatively coupled, may communicate data/messages with each other) via network 140. Network 140 may be a public network (e.g., the internet), a private network (e.g., a local area network (LAN) or wide area network (WAN)), or a combination thereof. In one embodiment, network 140 may include a wired or a wireless infrastructure, which may be provided by one or more wireless communications systems, such as a Wi-Fi hotspot connected with the network 140 and/or a wireless carrier system that can be implemented using various data processing equipment, communication towers (e.g., cell towers), etc. The network 140 may carry communications (e.g., data, message, packets, frames, etc.) between the local computing device 101 and the cloud storage system 113.

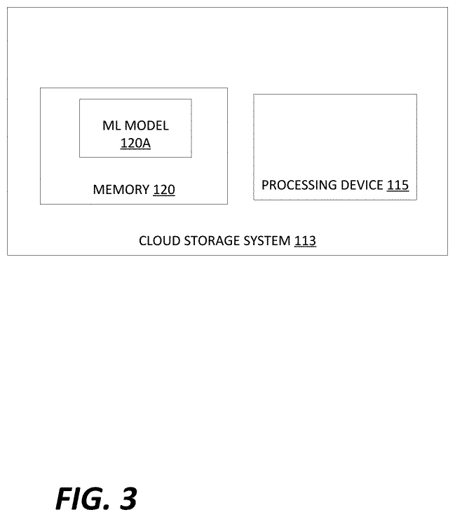

FIG. 3 illustrates the cloud storage system 113 in accordance with some embodiments of the present disclosure. As shown in FIG. 3, the cloud storage system 113 may be a computing device that includes hardware such as processing device 115 (e.g., processors, central processing units (CPUs)), memory 120 (e.g., random access memory (RAM)), storage devices (e.g., hard-disk drive (HDD), solid-state drive (SSD), etc.), and other hardware devices (e.g., sound card, video card, etc.). In some embodiments, memory 120 may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. Memory 120 may be configured for long-term storage of data and may retain data between power on/off cycles of the cloud storage system 113. The memory 120 may store the user data accumulated over time for the user as well as a multitude of other users. The user data accumulated over time for a particular user may form a time series health record for that particular user. The cloud storage system 113 may comprise any suitable type of computing device or machine that has a programmable processor including, for example, a server computer, a desktop computer, laptop computer, tablet computer, smartphone, etc. In some embodiments, the cloud storage system 113 may comprise a single computing device or may include multiple interconnected computing devices (e.g., multiple servers configured in a cloud storage cluster).

The cloud storage system 113 may provide third parties access to the user's user data. Examples of such third parties may include the user's physician, cardiac technicians, other cardiac specialists, and system administrators and managers. The third party may access the cloud storage system 113 via any appropriate type of computing device (e.g., personal computer, tablet computer, or smartphone) through network 140.

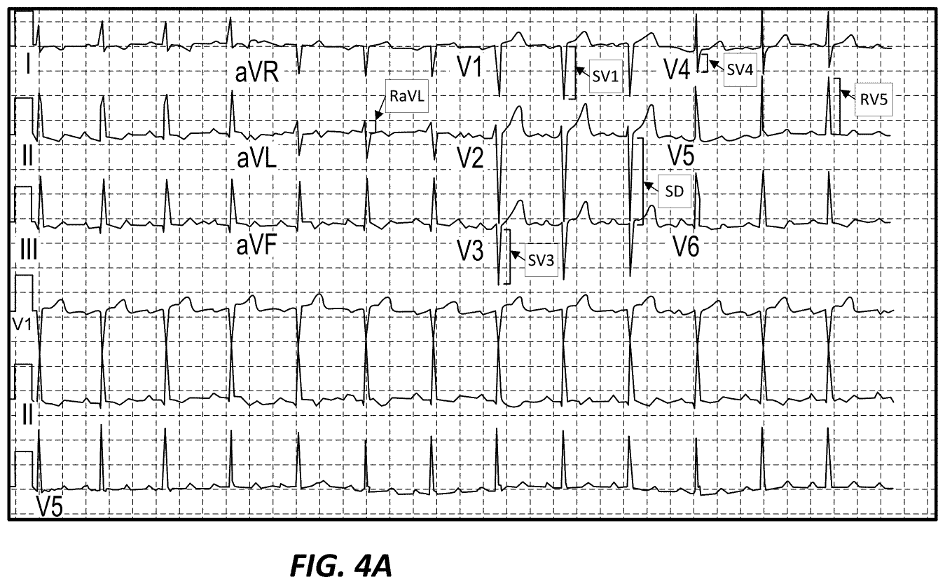

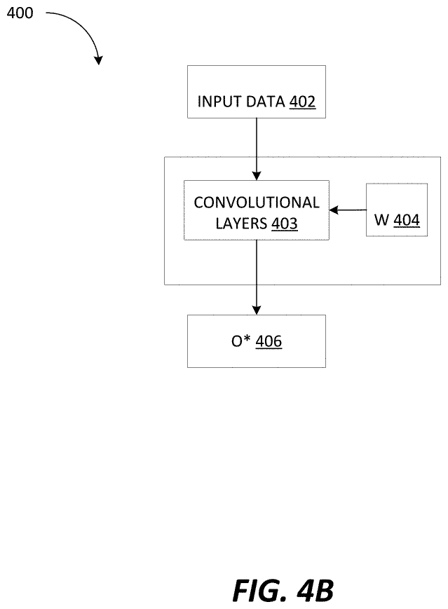

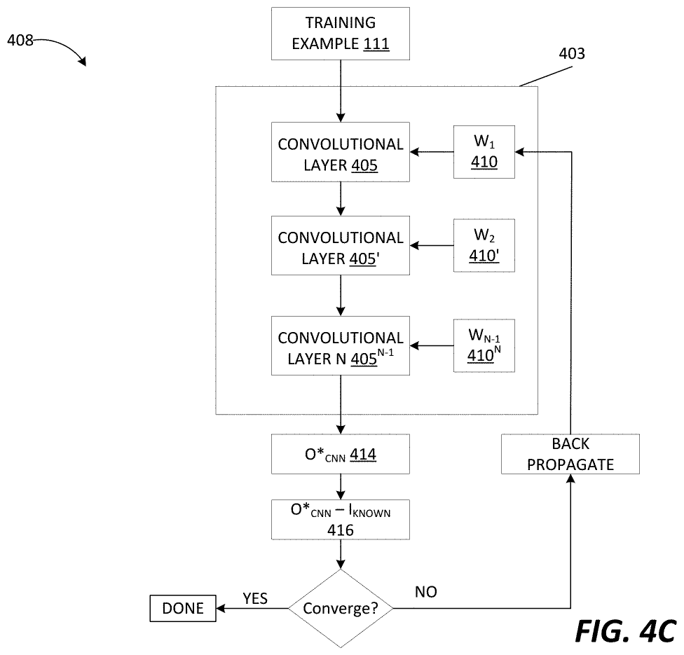

The memory 120 may further include a machine learning (ML) model 120A that is configured to analyze the ECG and blood pressure data of the user each time user data for the user is received and determine whether the user is experiencing LVH, and if so, the stage of progression of (i.e., the severity of) their LVH. The ML model 120A may analyze the ECG data using any appropriate ECG criteria for diagnosing LVH from an ECG analysis. With reference also to FIG. 4A which illustrates example ECG data of the first user, in one example, the ML model 120A may utilize the Cornell criteria, which provides that the R wave in the aVL lead is to be added to the S wave in the V3 lead (shown with black boxes in FIG. 4A). If the sum is greater than 28 millimeters (mm) in males or greater than 20 mm in females, this may be an indication that LVH is present according to the Cornell criteria. The ECG criteria used may further specify the severity of the LVH by defining ranges for the R wave, S wave, and the sum of both, and classifying users (e.g., as normal LVH or severe LVH) based on the range their R wave, S wave, and sum values fall into. Further examples of ECG criteria include the Sokolow-Lyon criteria, the Romhilt-Estes LVH Point Score System, and the modified Cornell criteria.

The ML model 120A may augment the result of the ECG data analysis with the blood pressure data. In analyzing the blood pressure data, the ML model 120A may utilize blood pressure criteria comprising a set of systolic and diastolic pressure ranges to classify users. More specifically, the ML model 120A may classify users with baseline systolic and diastolic BP values (<120/80 mm Hg) as normotensives, classify users with systolic BP from 120 to 139 mm Hg and diastolic BP from 80 to 89 mm Hg as prehypertensives, and classify users with systolic BP and diastolic BP values equal or higher than 140/90 mm Hg respectively as hypertensives. If the ML model 120A classifies the user as a hypertensive (e.g., more likely to suffer from LVH), it may adjust the result of the ECG analysis accordingly. For example, in cases where the ECG data analysis indicates a borderline case of LVH (e.g., R wave and S wave sum is 29 mm in a male user), if the ML model 120A determines that the user is hypertensive, it may strengthen the confidence of the determination that LVH is present. In another example, if the result of the ECG analysis indicates that the user is suffering from normal LVH, but is on the border of severe LVH, and the ML model 120A determines that the user is hypertensive, it may upgrade the severity classification to severe LVH. Similarly, if the ML model 120A classifies the user as a prehypertensive (e.g., having a slightly increased risk of suffering from LVH) it may adjust the result of the ECG analysis accordingly. Finally, if ML model 120A classifies the user as normotensive, (e.g., at the lowest risk of suffering from LVH), it may adjust the result of the ECG analysis accordingly. For example, in cases where the ECG data analysis indicates that the user is not suffering from LVH but is close to it (e.g., R wave and S wave sum is 28 mm in a male user), if the ML model 120A determines that the user is normotensive, it may strengthen the confidence of the determination that the user is not suffering from LVH. In another example, if the result of the ECG analysis indicates that the user is suffering from severe LVH, but is on the border of normal LVH, and the ML model 120A determines that the user is normotensive, it may downgrade the severity classification to normal LVH. The ML model 120A may then generate an LVH diagnosis based on the adjusted result of the ECG analysis.

By combining blood pressure and ECG data as described above, the ML model 120A more produce a more accurate diagnosis and may better account for noise from fluctuations in blood pressure data as well as ECG analysis results that indicate borderline cases. In some embodiments, the blood pressure data and the ECG data may have equal weight initially, and the ML model 120A may adjust the weight assigned to each component as user data of various other users is accumulated.

In some embodiments, the ML model 120A may augment its LVH diagnosis based on the user characteristics found in the user data, such as age, sex, race, ethnicity, relevant medical history, location, diet (e.g., food/drink habits), medication/drug consumption, exercise patterns, sleep/rest patterns, feelings of stress, anxiety, pain or other unusual or abnormal feelings, or any other user characteristic that may affect the first user's ECG and blood pressure data that is found in the user data. For example, upon determining that the sum of the first user's R wave and S wave is at or around 28 millimeters (e.g., a borderline case), the ML model 120A may determine that the first user is more likely to be suffering from LVH if their diet is poor and/or they have been feeling heightened stress and anxiety. Based on all of the above, the ML model 120A may output an LVH diagnosis indicating whether the first user is suffering from LVH and if so, the severity of the LVH.

In order to perform the above analysis, the ML model 120A may be trained using a training data set comprising user data from a variety of different users. Because the training data set may include user characteristics for each of the different users, the ML model 120A may further tailor any LVH diagnosis to the user based on user factors as discussed hereinabove. Through the training using the training data set, the ML model 120A may learn not only what combinations of ECG and blood pressure values are indicative of LVH (or particular stages/severities of LVH), but also which user characteristics (e.g., age, sex, race, ethnicity, and relevant medical history, location, diet (e.g., food/drink habits), medication/drug consumption, exercise patterns, sleep/rest patterns, feelings of stress, anxiety, pain or other unusual or abnormal feelings) are associated with and impact the determination of whether LVH is present in a variety of different users.

The ML model 120A may generate a diagnosis (comprising an indication of whether LVH is present and the severity of the LVH if it is present), which can be viewed by the user's physician. Upon receiving from the ML model 120A a diagnosis that the first user is suffering from LVH (and the severity), the physician may prescribe a course of treatment (i.e., an intervention) for the first user in order to mitigate the effects of the LVH. For example, the physician may prescribe LVH medication such as Angiotensin converting enzyme (ACE) inhibitors or beta blockers. Alternatively, or in addition, the physician may instruct the first user to avoid certain behaviors, habits, activities, foods, drinks, medications/drugs, etc. which are associated with/are contributing factors to the detection of LVH in the user. Further, the physician may instruct the first user to begin engaging in certain behaviors, habits, activities, foods, drinks, medications, drugs, etc. In some embodiments, the ML model 120A may prescribe the course of treatment, as described in further detail herein.

During the course of treatment, the first user may continue to record user data including ECG and blood pressure data (e.g., at the same or at different intervals than before the course of treatment) and these recordings may be transmitted by the local computing device 101 to the cloud storage system 113 and added to the first user's health record therein. User data recorded after a course of treatment has been prescribed may also be referred to as further user data. The ML model 120A may continue to analyze the first user's further user data to determine whether their ECG and blood pressure data indicate improvement (or worsening) in their LVH as discussed hereinabove. As the ML model 120A detects improvements in the ECG and blood pressure data, it may associate the course of treatment including prescribed LVH medication and/or modifications to behavior, habits, activities, diet, medications/drugs, etc. to the first user's improved LVH. Similarly, as the ML model 120A detects no change or a worsening in the ECG and blood pressure data, it may associate the course of treatment including prescribed LVH medication and/or modifications to behavior, habits, activities, diet, medications/drugs, etc. to the first user's unchanged or worsened LVH. The ML model 120A may perform this analysis for various users engaging in a variety of courses of treatment.

Over time, the ML model 120A may analyze the user data of various other users who are currently undergoing courses of treatment for LVH (i.e., further user data of the various other users). Some of these other users may have user characteristics similar to the first user's user characteristics (also referred to herein as similar users), and the ML model 120A may learn that certain courses of treatment are effective for similar users, while other courses of treatment are less effective or not effective at all for similar users. For example, the ML model 120A may observe that the first user, along with some similar users respond well (i.e., have ECG and blood pressure data indicating an improvement in their LVH) to ACE inhibitors. The ML model 120A may also observe that some other similar users did not respond well (i.e., have ECG and blood pressure data indicating no improvement or worsening of their LVH) to beta blockers.

The ML model 120A may also learn that similar users responded well to the behaviors, habits, activities, diets, medications/drugs, etc. which were prescribed by the physician as part of the course of treatment for the first user. The ML model 120A may further learn that similar users responded well to the elimination of behaviors, habits, activities, diets, medications, drugs, etc. which were prohibited by the course of treatment prescribed for the first user. The ML model 120A may also analyze user data for similar users whose course of treatment did not result in an improvement in (or worsened) their LVH and learn the LVH medications, as well as behaviors, habits, activities, diets, and medications/drugs etc. that do not impact or result in a worsening of the LVH in such similar users. As the ML model 120A continues to analyze the further user data of various other users, it may learn what courses of treatment are effective for different groups of users suffering from LVH.

Although discussed in terms of users who have similar user characteristics, for the purposes of identifying courses of treatment that are successful, the ML model 120A may define similar users based on any appropriate number of user characteristics, and may tailor courses of treatment appropriately. It should be noted that the ML model 120A may group users based on the user data it analyzes (i.e., the user data may guide the determination of similar users). For example, as the ML model 120A observes further user data for various users over time, it may determine that among users in the same age group, the successful course of treatment is different based on one or more of sex, race, ethnicity, and relevant medical history. The ML model 120A may observe that among users in the same age group, users of a first race and having a particular underlying health condition respond better to a first course of treatment, whereas users of second race and having no underlying health condition respond better to a second course of treatment. The ML model 120A may further determine that among the users of the second race who have no underlying health condition, male users respond better to the second course of treatment, while female users respond better to a third course of treatment which may correspond to e.g., the second course of treatment with certain dietary modifications. In this way, the ML model 120A may eventually personalize courses of treatment for individuals based on any number of particular user characteristics. By monitoring further user data, the ML model 120A may learn what courses of treatment work for individuals in a variety of different groups (regardless of how broadly or narrowly defined).

Once the ML model 120A has analyzed sufficient data to learn an appropriate course of treatment for different groups of users, it may begin recommending treatment plans to users in response to detecting that their user data indicates LVH. Based on a particular user's user data, the ML model 120A may identify a course of treatment to recommend (i.e., the LVH medication to prescribe, as well as behaviors, habits, activities, foods, drinks, medications/drugs, etc. to engage in and which to avoid) based on a course of treatment that has shown to be successful in treating LVH in other users that are similar to this particular user. The particular user may avoid a future healthcare issue caused by complications from LVH by modifying their behavior, habits, diet, exercise regimen, and/or by taking an LVH medication, as recommended by the ML model 120A.

Machine learning is well suited for continuous monitoring of one or multiple criteria to identify anomalies or trends, big and small, in input data as compared to training examples used to train the model. The ML model described herein may be trained on user data from a population of users, and/or trained on other training examples to suit the design needs for the model. Machine learning models that may be used with embodiments described herein include by way of example and not limitation: Bayes, Markov, Gaussian processes, clustering algorithms, generative models, kernel and neural network algorithms. Some embodiments utilize a machine learning model based on a trained neural network (e.g., a trained recurrent neural network (RNN) or a trained convolution neural network (CNN)).

FIG. 4A illustrates an example of a machine learning model 400 that could be used in conjunction with some embodiments of the present disclosure. The ML model 400 may comprise a trained CNN ML model that takes input data 402 (e.g., user data) into convolutional layers (aka hidden layers) 403, applies a series of trained weights or filters 404 to the input data 406 in each of the convolutional layers 403. The output of the first convolutional layer is an activation map (not shown), which is the input to the second convolution layer, to which a trained weight or filter (not shown) is applied, where the output of the subsequent convolutional layers results in activation maps that represent more and more complex features of the input data to the first layer. After each convolutional layer a non-linear layer (not shown) is applied to introduce non-linearity into the problem, which nonlinear layers may include an activation function such as tanh, sigmoid or ReLU. In some cases, a pooling layer (not shown) may be applied after the nonlinear layers, also referred to as a downsampling layer, which basically takes a filter and stride of the same length and applies it to the input, and outputs the maximum number in every sub-region the filter convolves around. Other options for pooling are average pooling and L2-norm pooling. The pooling layer reduces the spatial dimension of the input volume reducing computational costs and to control overfitting. The final layer(s) of the network is a fully connected layer, which takes the output of the last convolutional layer and outputs an n-dimensional output vector representing the quantity to be predicted, e.g., probabilities of LVH diagnosis and (if applicable) severity of LVH. This may result in predictive output 406 (O*), e.g., this user is likely suffering from stage 2 LVH. The trained weights 404 may be different for each of the convolutional layers 403, as will be described more fully below.

To achieve this real-world prediction/detection (e.g., user is suffering from stage 2 LVH), a neural network needs to be trained on known data inputs or training examples resulting in trained CNN 408. To train CNN 400, many different training examples (e.g., user data from users in various stages of LVH including no LVH) are input into the model. A skilled artisan in neural networks will fully understand the description above provides a somewhat simplistic view of CNNs to provide some context for the present discussion and will fully appreciate the application of any CNN alone or in combination with other neural networks or other entirely different machine learning models will be equally applicable and within the scope of some embodiments described herein.

FIG. 4B demonstrates training CNN 408. In FIG. 4B convolutional layers 403 are shown as individual hidden convolutional layers 405, 405′ to convolutional layer (405)ⁿ⁻¹ and the final nth layer is a fully connected layer. It will be appreciated that last layers may be more than one fully connected layer. Training example 411 is input into convolutional layers 403, a nonlinear activation function (not shown) and weights 410, 410′ through 410ⁿ are applied to training example 411 in series, where the output of any hidden layer is input to the next layer, and so on until the final nth fully connected layer (405)ⁿ⁻¹ produces output 414. Output or prediction 414 (*) is compared against training example 411 (e.g., LVH diagnosis performed with Echo) resulting in difference 416 between output or prediction 414 and training example 411 (also shown as I_{known} in FIG. 4B). If difference or loss 416 is less than some preset loss (e.g., output or prediction 414 predicts the user is suffering from LVH at the correct level of severity), the CNN is converged and considered trained. If the CNN has not converged, using the technique of backpropagation, weights 410 and 410′ through 410ⁿ are updated in accordance with how close the prediction is to the known input. The skilled artisan will appreciate that methods other than back propagation may be used to adjust the weights. The second training example (e.g., different user data) is input and the process is repeated again with the updated weights, which are then updated again and so on until the nth training example (e.g., nth user data) has been input. This is repeated over and over with the same n-training examples until the convolutional neural network (CNN) 400 is trained or converges on the correct outputs for the known inputs. Once CNN 408 is trained, weights 410, 410′ through 410′ are fixed and used in trained CNN 400, which are weights 404 as depicted in FIG. 4A. As explained, there are different weights for each convolutional layer 403 and for each of the fully connected layers. The trained CNN 400 or model is then fed user data to determine or predict that which it is trained to predict/identify (e.g., diagnose LVH based on user data), as described above. Any trained model, CNN, RNN, etc. may be trained further, i.e., modification of the weights may be permitted, with additional training examples or with predicted data output by the model which is then used as a training example. The machine learning model can be trained “offline”, e.g., trained once on a computational platform separate from the platform using/executing the trained model, and then transferred to that platform. Alternatively, embodiments described herein may periodically or continually update the machine learning model based on newly acquired training data. This updated training may occur on a separate computational platform which delivers the updated trained models to the platform using/executing the re-trained model over a network connection, or the training/re-training/update process may occur on the platform itself as new data is acquired. The skilled artisan will appreciate the CNN is applicable to data in a fixed array (e.g., a picture, character, word etc.) or a time sequence of data.

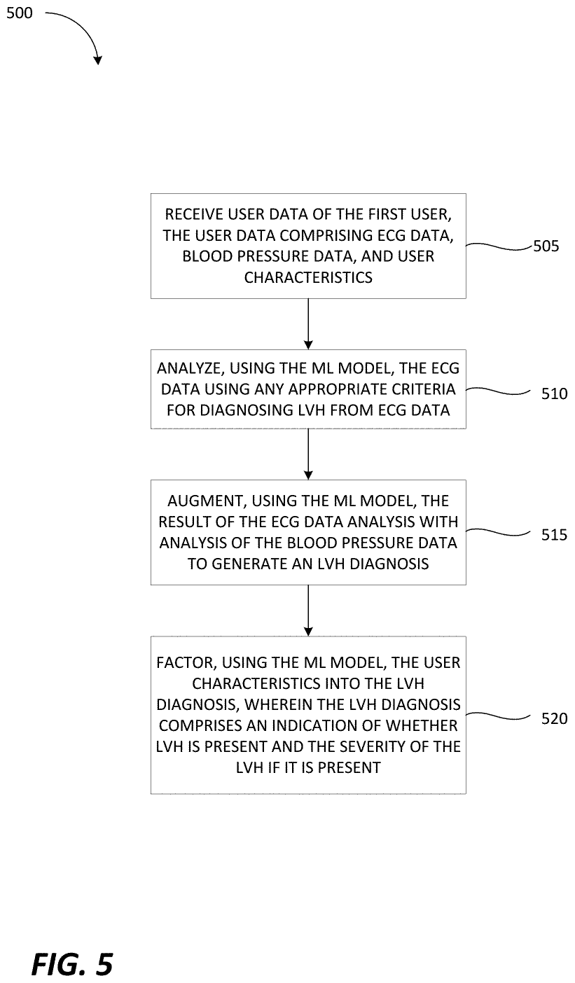

FIG. 5 is a flow diagram of a method 500 for diagnosing LVH based at least in part of ECG and blood pressure data, in accordance with some embodiments of the present disclosure. Method 500 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 500 may be performed by a computing device (e.g., cloud storage system 113 illustrated in FIG. 3).

Referring simultaneously to FIG. 3 as well, the method 500 begins at block 505, where the cloud storage system 113 may receive user data of the first user. The user data can be transmitted by the local computing device 101 to the cloud storage system 113 for storage and analysis. The transmission can be real-time, at regular intervals such as hourly, daily, weekly and/or any interval in between, or can be on demand.

The memory 120 may further include a machine learning (ML) model 120A that is configured to analyze the ECG and blood pressure data of the first user each time user data for the first user is received and determine whether the first user is experiencing LVH, and if so, the stage of progression of (i.e., the severity of) their LVH. At block 510, the ML model 120A may analyze the ECG data using any appropriate ECG criteria for diagnosing LVH from an ECG analysis. With reference also to FIG. 4A which illustrates example ECG data of the user, in one example, the ML model 120A may utilize the Cornell criteria, which provides that the R wave in the aVL lead is to be added to the S wave in the V3 lead (shown with black boxes in FIG. 4A). Then, if the sum is greater than 28 millimeters in males or greater than 20 mm in females, this may be an indication that LVH is present according to the Cornell criteria. The ECG criteria used may further specify the severity of the LVH by defining ranges for the R wave, S wave, and the sum of both, and classifying users (e.g., as normal LVH or severe LVH) based on the range their R wave, S wave, and sum values fall into. Further examples of ECG criteria include the Sokolow-Lyon criteria, the Romhilt-Estes LVH Point Score System, and the modified Cornell criteria.

At block 515, the ML model 120A may augment the result of the ECG data analysis with the blood pressure data. In analyzing the blood pressure data, the ML model 120A may utilize blood pressure criteria comprising a set of systolic and diastolic pressure ranges to classify users. More specifically, the ML model 120A may classify users with baseline systolic and diastolic BP values (<120/80 mm Hg) as normotensives, classify users with systolic BP from 120 to 139 mm Hg and diastolic BP from 80 to 89 mm Hg as prehypertensives, and classify users with systolic BP and diastolic BP values equal or higher than 140/90 mm Hg respectively as hypertensives. If the ML model 120A classifies the user as a hypertensive (e.g., more likely to suffer from LVH), it may adjust the result of the ECG analysis accordingly. For example, in cases where the ECG data analysis indicates a borderline case of LVH (e.g., R wave and S wave sum is 29 mm in a male user), if the ML model 120A determines that the user is hypertensive, it may strengthen the confidence of the determination that LVH is present. In another example, if the result of the ECG analysis indicates that the user is suffering from normal LVH, but is on the border of severe LVH, and the ML model 120A determines that the user is hypertensive, it may upgrade the severity classification to severe LVH. Similarly, if the ML model 120A classifies the user as a prehypertensive (e.g., having a slightly increased risk of suffering from LVH) it may adjust the result of the ECG analysis accordingly. Finally, if ML model 120A classifies the user as normotensive, (e.g., at the lowest risk of suffering from LVH), it may adjust the result of the ECG analysis accordingly. For example, in cases where the ECG data analysis indicates that the user is not suffering from LVH but is close to it (e.g., R wave and S wave sum is 28 mm in a male user), if the ML model 120A determines that the user is normotensive, it may strengthen the confidence of the determination that the user is not suffering from LVH. In another example, if the result of the ECG analysis indicates that the user is suffering from severe LVH, but is on the border of normal LVH, and the ML model 120A determines that the user is normotensive, it may downgrade the severity classification to normal LVH. The ML model 120A may then generate an LVH diagnosis based on the adjusted result of the ECG analysis.

By combining blood pressure and ECG data as described above, the ML model 120A more produce a more accurate diagnosis and may better account for noise from fluctuations in blood pressure data as well as ECG analysis results that indicate borderline cases. In some embodiments, the blood pressure data and the ECG data may have equal weight initially, and the ML model 120A may adjust the weight assigned to each component as user data of various other users is accumulated.

At block 520, in some embodiments the ML model 120A may augment its LVH diagnosis based on the user characteristics found in the user data, such as age, sex, race, ethnicity, relevant medical history, location, diet (e.g., food/drink habits), medication/drug consumption, exercise patterns, sleep/rest patterns, feelings of stress, anxiety, pain or other unusual or abnormal feelings, or any other user characteristic that may affect the first user's ECG and blood pressure data that is found in the user data. For example, upon determining that the sum of the first user's R wave and S wave is at or around 28 millimeters (e.g., a borderline case), the ML model 120A may determine that the first user is more likely to be suffering from LVH if their diet is poor and/or they have been feeling heightened stress and anxiety. Based on all of the above, the ML model 120A may output an LVH diagnosis indicating whether the first user is suffering from LVH and if so, the severity of the LVH.

In order to perform the above analysis, the ML model 120A may be trained using a training data set comprising user data (including ECG, blood pressure, and user data) from a variety of different users. Because the training data set may include user characteristics for each of the different users, the ML model 120A may further tailor any LVH diagnosis to the user based on user factors as discussed hereinabove. Through the training using the training data set, the ML model 120A may learn not only what combinations of ECG and blood pressure values are indicative of LVH (and the particular stage/severity of LVH), but also which user characteristics (e.g., age, sex, race, ethnicity, relevant medical history, location, diet (e.g., food/drink habits), medication/drug consumption, exercise patterns, sleep/rest patterns, feelings of stress, anxiety, pain or other unusual or abnormal feelings) are associated with and impact the determination of whether LVH is present (and there severity of the LVH if it is present) in a variety of different users.

The ML model 120A may generate the diagnosis (comprising an indication of whether LVH is present and the severity of the LVH if it is present), which can be viewed by the first user's physician. Upon receiving from the ML model 120A a diagnosis that the first user is suffering from LVH (and the severity), the physician may prescribe a course of treatment (i.e., an intervention) for the first user in order to mitigate the effects of the LVH. For example, the physician may prescribe LVH medication such as Angiotensin converting enzyme (ACE) inhibitors or beta blockers. Alternatively, or in addition, the physician may instruct the first user to avoid certain behaviors, habits, activities, foods, drinks, medications/drugs, etc. which are associated with/are contributing factors to the detection of LVH in the user. Further, the physician may instruct the first user to begin engaging in certain behaviors, habits, activities, foods, drinks, medications, drugs, etc. In some embodiments, the ML model 120A may prescribe the course of treatment, as described in further detail herein.

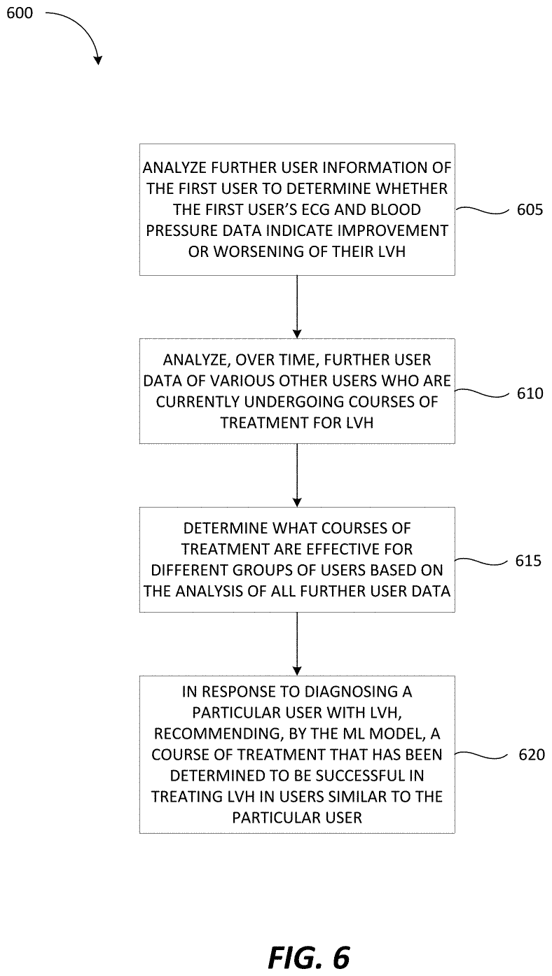

FIG. 6 is a flow diagram of a method 600 of identifying optimal courses of treatment for reducing LVH, in accordance with some embodiments of the present disclosure. Method 600 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 600 may be performed by a computing device (e.g., cloud storage system 113 illustrated in FIG. 3).

During the course of treatment, the first user may continue to record user data including ECG and blood pressure data (e.g., at the same or at different intervals than before the course of treatment) and these recordings may be transmitted by the local computing device 101 to the cloud storage system 113 and added to the first user's health record therein. User data recorded after a course of treatment has been prescribed may also be referred to as further user data. At block 605, the ML model 120A may continue to analyze the first user's further user data to determine whether their ECG and blood pressure data indicate improvement (or worsening) in their LVH as discussed hereinabove. As the ML model 120A detects improvements in the ECG and blood pressure data, it may associate the course of treatment including prescribed LVH medication and/or modifications to behavior, habits, activities, diet, medications/drugs, etc. to the user's improved LVH. Similarly, as the ML model 120A detects no change or a worsening in the ECG and blood pressure data, it may associate the course of treatment including prescribed LVH medication and/or modifications to behavior, habits, activities, diet, medications/drugs, etc. to the first user's unchanged or worsened LVH. The ML model 120A may perform this analysis for various users engaging in a variety of courses of treatment.

Over time, the ML model 120A may analyze, at block 610, the user data of various other users who are currently undergoing courses of treatment for LVH (i.e., further user data of the various other users). Some of these other users may have user characteristics similar to first user's user characteristics (also referred to herein as similar users), and the ML model 120A may learn that certain courses of treatment are effective for similar users, while other courses of treatment are less effective or not effective at all for similar users. For example, the ML model 120A may observe that the first user, along with some similar users respond well (i.e., have ECG and blood pressure data indicating an improvement in their LVH) to ACE inhibitors. The ML model 120A may also observe that some other similar users did not respond well (i.e., have ECG and blood pressure data indicating no improvement or worsening of their LVH) to beta blockers.

The ML model 120A may also learn that similar users responded well to the behaviors, habits, activities, diets, medications/drugs, etc. which were prescribed by the physician as part of the course of treatment for first user. The ML model 120A may further learn that similar users responded well to the elimination of behaviors, habits, activities, diets, medications, drugs, etc. which were prohibited by the course of treatment prescribed for the first user. The ML model 120A may also analyze user data for similar users whose course of treatment did not result in an improvement in (or worsened) their LVH and learn the LVH medications, as well as behaviors, habits, activities, diets, and medications/drugs etc. that do not affect, or result in a worsening of the LVH in such similar users. At block 615, as the ML model 120A continues to analyze the user data of various other users, it may learn what courses of treatment are effective for different groups of users.

Although discussed in terms of users who have similar user characteristics, for the purposes of identifying courses of treatment that are successful, the ML model 120A may define similar users based on any appropriate number of user characteristics, and may tailor courses of treatment appropriately. It should be noted that the ML model 120A may group similar users based on the user data it analyzes (i.e., the user data may guide the determination/groupings of similar users). For example, as the ML model 120A observes further user data for various users over time, it may determine that among users in the same age group, the successful course of treatment is different based on one or more of sex, race, ethnicity, and relevant medical history. The ML model 120A may observe that among users in the same age group, users of a first race and having a particular underlying health condition respond better to a first course of treatment, whereas users of second race and having no underlying health condition respond better to a second course of treatment. The ML model 120A may further determine that among the users of the second race who have no underlying health condition, male users respond better to the second course of treatment, while female users respond better to a third course of treatment which may correspond to e.g., the second course of treatment with certain dietary modifications. In this way, the ML model 120A may eventually personalize courses of treatment for individuals based on any number of particular user characteristics. By monitoring further user data, the ML model 120A may learn what courses of treatment work for individuals in a variety of different groups (regardless of how broadly or narrowly defined).

Once the ML model 120A has analyzed sufficient data to learn an appropriate course of treatment for a variety of different users, at block 620, it may begin recommending treatment plans to users in response to detecting that their user data indicates LVH. Based on a particular user's user data, the ML model 120A may identify a course of treatment to recommend (i.e., the LVH medication to prescribe, as well as behaviors, habits, activities, foods, drinks, medications/drugs, etc. to engage in and which to avoid) based on a course of treatment that has shown to be successful in treating LVH in other users that are similar to this particular user (e.g., based on user characteristics). The particular user may avoid a future healthcare issue caused by complications from LVH by modifying their behavior, habits, diet, exercise regimen, and/or by taking an LVH medication, as recommended by the ML model 120A.

Machine learning is well suited for continuous monitoring of one or multiple criteria to identify anomalies or trends, big and small, in input data as compared to training examples used to train the model. The ML model described herein may be trained on user data from a population of users, and/or trained on other training examples to suit the design needs for the model. Machine learning models that may be used with embodiments described herein include by way of example and not limitation: Bayes, Markov, Gaussian processes, clustering algorithms, generative models, kernel and neural network algorithms. Some embodiments utilize a machine learning model based on a trained neural network (e.g., a trained recurrent neural network (RNN) or a trained convolution neural network (CNN)).

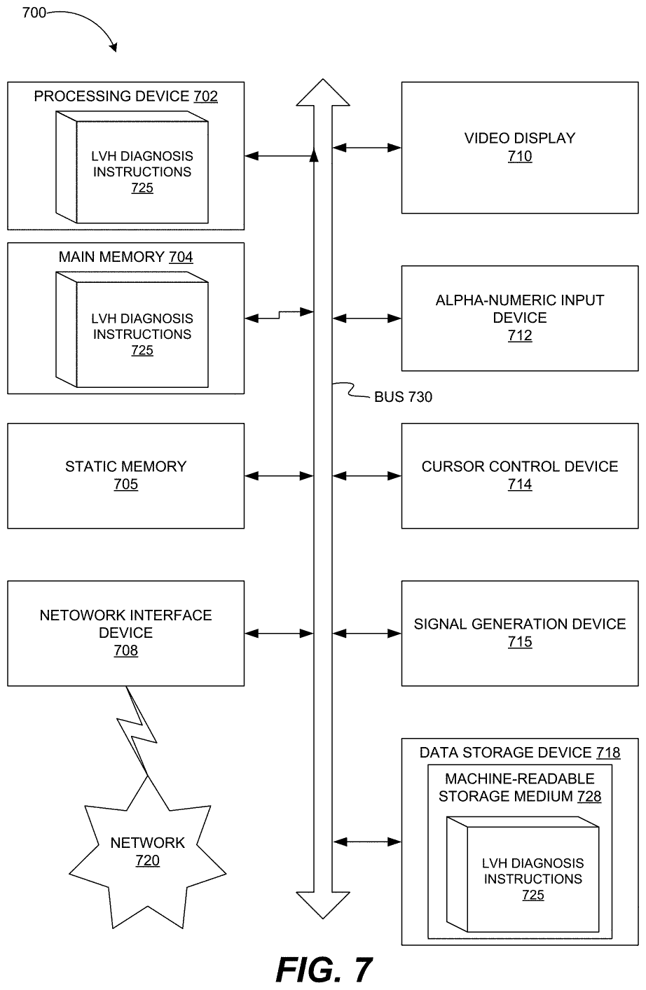

FIG. 7 illustrates a diagrammatic representation of a machine in the example form of a computer system 700 within which a set of instructions, for causing the machine to perform any one or more of the methodologies discussed herein for diagnosing LVH based at least in part on ECG and blood pressure data.

In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 700 may be representative of a server.

The exemplary computer system 700 includes a processing device 702, a main memory 704 (e.g., read-only memory (ROM), flash memory, dynamic random access memory (DRAM), a static memory 706 (e.g., flash memory, static random access memory (SRAM), etc.), and a data storage device 718, which communicate with each other via a bus 730. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Computing device 700 may further include a network interface device 708 which may communicate with a network 720. The computing device 700 also may include a video display unit 710 (e.g., a liquid crystal display (LCD) or a cathode ray tube (CRT)), an alphanumeric input device 712 (e.g., a keyboard), a cursor control device 714 (e.g., a mouse) and an acoustic signal generation device 716 (e.g., a speaker). In one embodiment, video display unit 710, alphanumeric input device 712, and cursor control device 714 may be combined into a single component or device (e.g., an LCD touch screen).

Processing device 702 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. The processing device 702 is configured to execute LVH diagnosis instructions 725, for performing the operations and steps discussed herein.

The data storage device 715 may include a machine-readable storage medium 728, on which is stored one or more sets of LVH diagnosis instructions 725 (e.g., software) embodying any one or more of the methodologies of functions described herein. The LVH diagnosis instructions 725 may also reside, completely or at least partially, within the main memory 704 or within the processing device 702 during execution thereof by the computer system 700; the main memory 704 and the processing device 702 also constituting machine-readable storage media. The LVH diagnosis instructions 725 may further be transmitted or received over a network 720 via the network interface device 708.

While the machine-readable storage medium 728 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof

What is claimed is:

1. A method comprising:
receiving user data of a first user, the user data comprising electrocardiogram (ECG) data and blood pressure data of the first user;
analyzing, by a processing device executing a machine learning (ML) model, the user data to determine whether left ventricle hypertrophy (LVH) is present in the first user based on the ECG data and the blood pressure data; and
in response to determining that the LVH is present in the first user, outputting an indication that the LVH is present in the first user and a severity of the LVH in the first user.

2. The method of claim 1, wherein the user data of the first user further comprises characteristics of the first user and the ML model determines whether the LVH is present in the first user based further on the characteristics of the first user.

3. The method of claim 1, wherein determining whether the LVH is present in the first user comprises:
comparing the ECG data to ECG criteria comprising a set of ranges, wherein the set of ranges indicate whether the LVH is present and a severity of the LVH if it is present;
comparing the blood pressure data to blood pressure criteria defining a set of systolic and diastolic pressure ranges, each of the set of systolic and diastolic pressure ranges indicating a likelihood that the LVH is present; and
determining whether the LVH is present in the first user and a severity of the LVH if it is present, based on a range that the ECG data is within and a systolic and diastolic pressure range that the blood pressure data is within.

4. The method of claim 1, further comprising:
monitoring, by the ML model, further user data of the first user, the further user data captured while the first user is undergoing a course of treatment from among a plurality of courses of treatment for LVH; and
monitoring, by the ML model, further user data of each of a plurality of second users, further user data of each second user captured while the second user is undergoing one of the plurality of courses of treatment for LVH.

5. The method of claim 4, further comprising:
identifying, based on the monitoring of the further user data of the first user and the further user data of the plurality of second users, one or more of the plurality of courses of treatment that are successful in reducing LVH, each of the one or more courses of treatment successfully reducing LVH in a subset of the plurality of second users.

6. The method of claim 5, further comprising:
in response to determining that LVH is present in a third user based on user data of the third user, recommending, by the ML model, a course of treatment of the one or more courses of treatment based on the user data of the third user.

7. The method of claim 1, wherein the ML model comprises a neural network model.

8. A system comprising:
an electrocardiogram (ECG) monitor to record ECG data of a first user;
a blood pressure monitor to record blood pressure data of the first user; and
a cloud storage system to:
receive user data of a first user, the user data comprising the electrocardiogram (ECG) data and the blood pressure data of the first user;
analyze, by a machine learning (ML) model, the user data to determine whether left ventricle hypertrophy (LVH) is present in the first user based on the ECG data and the blood pressure data; and
in response to determining that the LVH is present in the first user, output an indication that the LVH is present in the first user and a severity of the LVH in the first user.

9. The system of claim 8, wherein the user data of the first user further comprises characteristics of the first user and the ML model determines whether the LVH is present in the first user based further on the characteristics of the first user.

10. The system of claim 8, wherein to determine whether the LVH is present in the first user, the cloud storage system is to:
compare the ECG data to ECG criteria comprising a set of ranges, wherein the set of ranges indicate whether the LVH is present and a severity of the LVH if it is present;
compare the blood pressure data to blood pressure criteria defining a set of systolic and diastolic pressure ranges, each of the set of systolic and diastolic pressure ranges indicating a likelihood that the LVH is present; and
determine whether the LVH is present in the first user and a severity if it is, based on a range that the ECG data is within and a systolic and diastolic pressure range that the blood pressure data is within.

11. The system of claim 8, wherein the cloud storage system is further to:
monitor, by the ML model, further user data of the first user, the further user data captured while the first user is undergoing a course of treatment from among a plurality of courses of treatment for LVH; and
monitor, by the ML model, further user data of each of a plurality of second users, further user data of each second user captured while the second user is undergoing one of the plurality of courses of treatment for LVH.

12. The system of claim 11, wherein the cloud storage system is further to:
identify, based on the monitoring of the further user data of the first user and the further user data of the plurality of second users, one or more of the plurality of courses of treatment that are successful in reducing LVH, each of the one or more courses of treatment successfully reducing LVH in a subset of the plurality of second users.

13. The system of claim 12, wherein the cloud storage system is further to:
in response to determining that LVH is present in a third user based on user data of the third user, recommending, by the ML model, a course of treatment of the one or more courses of treatment based on the user data of the third user.

14. The system of claim 8, wherein the ML model comprises a neural network model.

15. A non-transitory computer-readable medium having instructions stored thereon which, when executed by a processing device, cause the processing device to:
receive user data of a first user, the user data comprising electrocardiogram (ECG) data and blood pressure data of the first user;
analyze, by the processing device executing a machine learning (ML) model, the user data to determine whether left ventricle hypertrophy (LVH) is present in the first user based on the ECG data and the blood pressure data; and
in response to determining that the LVH is present in the first user, output an indication that the LVH is present in the first user and a severity of the LVH in the first user.

16. The non-transitory computer-readable medium of claim 15, wherein the user data of the first user further comprises characteristics of the first user and the ML model determines whether the LVH is present in the first user based further on the characteristics of the first user.

17. The non-transitory computer-readable medium of claim 15, wherein to determine whether the LVH is present in the first user, the processing device is to:
compare the ECG data to ECG criteria comprising a set of ranges, wherein the set of ranges indicate whether the LVH is present and a severity of the LVH if it is present;
compare the blood pressure data to blood pressure criteria defining a set of systolic and diastolic pressure ranges, each of the set of systolic and diastolic pressure ranges indicating a likelihood that the LVH is present; and
determine whether the LVH is present in the first user and a severity if it is, based on a range that the ECG data is within and a systolic and diastolic pressure range that the blood pressure data is within.

18. The non-transitory computer-readable medium of claim 15, wherein the processing device is further to:
monitor, by the ML model, further user data of the first user, the further user data captured while the first user is undergoing a course of treatment from among a plurality of courses of treatment for LVH; and
monitor, by the ML model, further user data of each of a plurality of second users, further user data of each second user captured while the second user is undergoing one of the plurality of courses of treatment for LVH.

19. The non-transitory computer-readable medium of claim 18, wherein the processing device is further to:
identify, based on the monitoring of the further user data of the first user and the further user data of the plurality of second users, one or more of the plurality of courses of treatment that are successful in reducing LVH, each of the one or more courses of treatment successfully reducing LVH in a subset of the plurality of second users.

20. The non-transitory computer-readable medium of claim 19, wherein the processing device is further to:
in response to determining that LVH is present in a third user based on user data of the third user, recommending, by the ML model, a course of treatment of the one or more courses of treatment based on the user data of the third user.
